(19) United States
(12) Patent Application Publication
(10) Pub. No.: US 2007/0086962 A1
(43) Pub. Date: Apr. 19, 2007

(54) Stable sunscreen composition

(75) Inventors: Prasun Bandyopadhyay, Bangalore (IN); Kritika Pershad, Hyderabad (IN); Amitava Pramanik, Bangalore (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/581,022

(22) Filed: Oct. 13, 2006

(30) Foreign Application Priority Data
Oct. 14, 2005 (IN) 1286/MUM/2005
Mar. 9, 2006 (EP) EP06251276

(51) Int. Cl. A61K 8/37

(52) U.S. Cl. 424/59

(57) ABSTRACT

A stable sunscreen composition comprising: (i) an organic UV-A sunscreen; (ii) an organic UV-B sunscreen; and (iii) cosmetically acceptable vehicle wherein said UV-A sunscreen and UV-B sunscreen are kept isolated from each other by encapsulating one of said sunscreens in solid lipid encapsulate, the solid lipid encapsulate comprising a lipid.

### TECHNICAL FIELD

The invention relates to sunscreen compositions where the organic sunscreens are so formulated as to display enhanced stability.

### BACKGROUND AND PRIOR ART

Sunscreens have been used in cosmetic compositions for topical application for a long time. Sunscreens are added to skin cosmetic compositions to block ultra-violet (UV) radiation from affecting the skin. Inorganic sunscreens act by physically blocking some or all of the UV rays causing the rays to be reflected or scattered away from the skin surface. Organic sunscreens act by absorbing some or all of the UV radiation thereby ensuring that the UV rays do not reach the skin surface. UV rays are generally classified as UV-A or UV-B rays. UV-A generally covers the UV wavelength range from 320 to 400 nm while UV-B covers the 280 to 320 nm range. While damage to the skin due to exposure to UV-A rays have been reported to cause irritation, redness and sunburns, exposure to UV-B rays causes more severe irritation, redness and sunburns in addition to erythema. Hence sunscreen formulations have generally been formulated with a combination of UV-A and UV-B sunscreens.

It is well known that the efficacy of organic sunscreens to block UV-rays reduces after a period of exposure to UV rays. Further it has been reported that many organic sunscreens interact with each other when applied on the skin and exposed to UV-radiation, thereby reducing their efficacy over time. Such compositions are therefore not very stable and thus are not very effective in blocking UV-rays when applied on the skin. Further, when such compositions are used and then exposed to sunlight or UV-rays, the period over which the skin is actually protected is very short, amounting to a few minutes, sometimes as low as from 10 to 30 minutes.

There has been some efforts in improving the stability of sunscreens when formulated together. It has been found that some classes of sunscreens when formulated with another class improves the stability. EP 780 119 A (Givaudan, 1997) describes a photostable cosmetic light-screening composition comprising essentially a dibenzoyl methane type UV-A screening agent and a α-cyano-β,β-diphenyl acylate stabiliser in specified amounts and ratios.

U.S. Pat. No. 6,436,375 (Sol-Gel Tech, 2002) describes a method of preparing a sunscreen composition with improved photostability that contains at least two sunscreen active ingredients, which are photo-unstable when formulated together, which method comprises the steps of separating the two ingredients from each other by microencapulating at least one of said ingredients in sol-gel microcapsules.

While some methods of enhancing the stability of organic sunscreens have been reported, there exists a need to develop compositions that provide enhanced stability so that the skin is protected against the harmful effects of UV radiation for longer periods of time.

### OBJECTS OF THE INVENTION

It is thus an object of the invention to provide for a sunscreen composition comprising an organic UVA sunscreen and an organic UVB susncreen that have enhanced stability during use.

It is another object of the invention to provide for a sunscreen composition that, while having enhanced stability is prepared using conventional cosmetic ingredients that are economically and widely available thereby ensuring a low cost to the consumer.

It is another object of the invention to provide for a sunscreen composition that while having enhanced stability and low cost, can be prepared by a simple and cost-effective process.

### SUMMARY OF THE INVENTION

According to one aspect of the invention there is provided a stable sunscreen composition comprising:
(i) an organic UV-A sunscreen;
(ii) an organic UV-B sunscreen; and
(iii) cosmetically acceptable vehicle

Wherein said UV-A sunscreen and UV-B sunscreen are kept isolated by encapsulating one of said sunscreens in solid lipid encapsulate.

A particularly preferred organic UV-A sunscreen is a dibenzoyl methane derivative. A particularly preferred UV-B sunscreen is octyl methoxycinnamate.

According to another aspect of the invention there is provided a process for preparation of the sunscreen composition of the invention comprising the steps of:
(a) preparing a slurry by mixing the sunscreen to be encapsulated with the lipid at a temperature in the range of 60 to 90° C.;
(b) cooling said slurry to a temperature in the range of 30 to 50° C.;
(c) preparing a base by mixing the cosmetically acceptable vehicle and the sunscreen to be unencapsulated, and
(d) mixing the cooled slurry of step (b) with the base of step (c), at a temperature in the range of 30 to 50° C. to prepare the sunscreen composition.

### DETAILED DESCRIPTION OF THE INVENTION

UV-A Sunscreen

The invention provides for a stable sunscreen composition. The composition essentially comprises a solid lipid encapsulate which comprises an organic UV-A sunscreen or an organic UV-B sunscreen. Alternately, the composition may comprise UV-A sunscreen and UV-B sunscreen, both of which may be separately encapsulated. The UV-A sunscreen is preferably a dibenzoyl methane derivative. The most preferred dibenzoyl methane derivative is 4-tert-butyl 4′-methoxy dibenzoyl methane which is availabe under the brand name Parsol 1789 from Givaudan. Other dibenzoyl methane derivatives which may be included in the solid lipid encapsulate of the invention include 2,4-dimethyl-4′-methoxy dibenzoylmethane, 2-methyl-5-tert-butyl-4′-methoxy dibenzoylmethane, 4,4′-diisopropyl-dibenzoylmethane and 2,4-dimethyl dibenzoylmethane. The UV-A suncreen is preferably present in an amount in the range of 0.1 to 5% by weight of the composition.

Solid Lipid Encapsulate

The solid lipid encapsulate is preferably a lipid which may be a fatty acid, a fatty acid ester, fatty alcohols or natural/synthetic waxes. Suitable fatty acid esters include e.g. mono-, di- or triglyceride. Particularly preferred lipids are fatty acid or fatty acid ester. It is desirable that the lipid which forms the solid lipid encapsulate has a melting point higher than room temperature. Thus the melting point of the lipid is preferably greater than 30° C., more preferably greater than 40° C. Especially suitable lipids are glycerol monostearate, stearic acid or cetyl palmitate. The solid lipid encapsulate is preferably present in an amount in the range of 1 to 50%, preferably 5 to 30% by weight of the composition.

Antioxidant

The stability of the sunscreens in the composition is seen to be enhanced when the solid lipid encapsulate comprises an antioxidant. Preferred antioxidants for use in the solid lipid encapsulate of the invention are tocopherol acetate, butyl hydoxy toluene, α-tocopherol, or C₈-C₁₈ fatty acid of alpha hydroxy acid. Especially preferred antioxidants are tocopherol acetate, butyl hydoxy toluene or ascorbyl palmitate. When an antioxidant is present in the solid lipid encapsulate, it is present in an amount in the range of 0.1 to 5%, preferably 0.1 to 2% by weight of the composition.

UV-B Sunscreen

The sunscreen composition of the invention comprises an organic UV-B sunscreen. Maximum sunscreen stability is obtained when the organic UV-B sunscreen is not present in the solid lipid encapsulate, but is included in the composition, outside of the solid lipid encapsulate. Alternately, the UV-B sunscreen is encapsulated in the solid lipid encapsulate and the UV-A sunscreen is present in the composition, outside of the encapsulate. Preferred UV-B sunscreens include octyl methoxycinnamate (2-ethylhexyl 4-methoxycinnamate). The organic UV-B sunscreen is preferably present in an amount in the range of 0.1 to 5% by weight of the composition.

Optional Ingredients

When an antioxidant is used in the solid lipid encapsulate of the sunscreen composition, an emulsifier is used to emulsify the antioxidant in water for incorporation in the composition of the invention. The emulsifier is preferably a non-ionic surfactant. Suitable non-ionic surfactants are chosen from the group comprising polyethylene sorbitan fatty acid esters, polyethylene alkyl ethers, polyethylene alkyl esters or sucrose esters. When present, the emulsifier is present in an amount in the range of 0.5 to 10% by weight of the composition. A skin lightening agent is preferably present in the cosmetic composition of the invention. Preferable skin lightening agents include niacinamide, aloe extract, ammonium lactate, azelaic acid, kojic acid, lactic acid, linoleic acid, magnesium ascorbyl phosphate, 5-octanoyl salicylic acid, 2,4-resorcinol derivatives, 3,5-resorcinol derivatives, salicylic acid, 3,4,5-trihydroxybenzyl derivatives, Vitamin B6, Vitamin A, Vitamin C and mixtures thereof. An especially preferred skin lightening agent is niacinamide. Skin lightening agent is preferably present from 0.1% to 10%, more preferably from 0.1% to 5%, by weight of the cosmetic composition.

Cosmetically Acceptable Vehicle

The cosmetic composition comprises an acceptable vehicle to act as a diluant, dispersant or carrier for the actives present in the composition, so as to facilitate their distribution when the composition is applied to the skin.

Cosmetically acceptable vehicle, other than water, can include liquid or solid emollients, solvents, humectants, thickeners and powders. Examples of each of these types of additional materials, which can be used singly or as mixtures, are as follows:

Emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate;

Propellants, such as propane, butane, isobutane, dimethyl ether, carbon dioxide, nitrous oxide;

Solvents, such as ethyl alcohol, isopropanol, acetone, ethylene glycol monoethyl ether, diethylene glycol monobutyl ether, diethylene glycol monoethyl ether;

Powders, such as chalk, talc, Fullers earth, kaolin, starch, gums, colloidal silica sodium polyacrylate, tetraalkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

These additional materials are preferably present from 10 to 99.9%, preferably from 50 to 99% by weight of the cosmetic composition, and can, in the absence of other cosmetic adjuncts, form the balance of the composition.

Optional Skin Benefit Agents

The composition of the invention may include other sunscreens or sunblock agent such as 2-hydroxy-4-methoxybenzophenone, octyldimethyl-p-aminobenzoic acid and mixtures thereof. Inorganic sunblocks are also preferably used in the present invention. These include, for example, zinc oxide, iron oxide, silica, such as fumed silica, and titanium dioxide. Ultrafine titanium dioxide in either of its two forms, namely water-dispersible titanium dioxide and oil-dispersible titanium dioxide is especially suitable for the invention. Water-dispersible titanium dioxide is ultra-fine titanium dioxide, the particles of which are non-coated or which are coated with a material to impart a hydrophilic surface property to the particles. Examples of such materials include aluminium oxide and aluminium silicate. Oil-dispersible titanium dioxide is ultrafine titanium dioxide, the particles of which exhibit a hydrophobic surface property, and which, for this purpose, can be coated with metal soaps such as aluminium stearate, aluminium laurate or zinc stearate, or with organosilicone compounds.

By “ultrafine titanium dioxide” is meant particles of titanium dioxide having an average particle size of less than 100 nm, preferably 70 nm or less, more preferably from 10 to 40 nm and most preferably from 15 to 25 nm.

Ultrafine titanium dioxide is the preferred inorganic sunblock agent. The total amount of sunblock that is preferably incorporated in the composition according to the invention is from 0.1 to 5% by weight of the composition.

Optional Cosmetic Ingredients

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include: antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents and skin healing agents.

Process

The invention provides a process for preparation of the sunscreen composition of the invention comprising the steps of:
(a) preparing a slurry by mixing the sunscreen to be encapsulated with the lipid at a temperature in the range of 60 to 90° C.
(b) cooling said slurry to a temperature in the range of 30 to 50° C.;
(c) preparing a base by mixing the cosmetically acceptable vehicle and the sunscreen to be unencapsulated; and
(d) mixing the cooled slurry of step (b) with the base of step (c) at a temperature in the range of 30 to 50° C. to prepare the sunscreen composition.

It is preferred that an emulsion comprising antioxidant and emulsifier in water is mixed with said slurry at a temperature in the range of 60 to 90° C. The slurry and emulsion are preferably mixed at high shear. The rpm of mixing is preferably from 1000 to 3000 rpm.

The base is preferably prepared at a temperature in the range of 55 to 90° C.

The base and the cooled slurry are preferably mixed at low shear. The rpm may be in the range of 200 to 1000 rpm.

The invention will now be illustrated with reference to the following non-limiting examples.

### EXAMPLES

### Examples 1, 2, and Comparative Example A

### Stability of the Compositions where the Sunscreens are Encapsulated as Compared to Prior Art

### Example 1

UV-A sunscreen butyl methoxy dibenzoyl methane (Parsol 1789) was encapsulated in a solid lipid encapsulate using cetyl palmitate as the lipid. A slurry of Parsol 1789 was prepared by mixing in molten cetyl palmitate at 70° C. at 2000 rpm to prepare a slurry. Non-ionic surfactant (Tween 80) and water were mixed and heated to 70° C. at 2000 rpm to prepare an emulsion. The slurry and the emulsion were mixed together at 2000 rpm and then cooled to 40° C. to form the lipid encapsulate of Parsol 1789. UV-B sunscreen octyl methoxy cinnamate (Parsol MCX) was dispersed in water at 40° C. before mixing with the slurry 40° C. at 400 rpm.

The composition was then cooled to 25° C. The compositions had 1% of Parsol 1789 and 1% of Parsol MCX with the cetyl palmitate at 20%, Tween 80 at 4%, the rest being water.

### Example 2

A composition as per Example 1 was prepared except that the same amount of Parsol MCX was encapsulated in the solid lipid encapsulate, while Parsol 1789 was unencapsulated by including in the emulsion.

### Comparative Example A

A conventional sunscreen composition was prepared where both Parsol MCX and Parsol 1789 were unencapsulated. Procedure as per Example 1 or 2 was followed except that both the sunscreens were mixed with in the emulsion.

Test Method

Equal amounts of the compositions of Examples 1, 2 and A (10 milligrams) were taken and spread on several glass slides and exposed to the sun at the same time. A sample glass slide was taken at different times of exposure from zero to 120 minutes. The samples were dissolved in methanol and the absorbancy of the sample was measured at 362 nm (corresponding to the peak absorption wavelength of Parsol 1789). The measurement was carried out using a Perkin Elmer (Lamda 900) UV/VIS/NIR spectrophotometer. The % absorbancy remaining which is an indicator of the stability of the sample was calculated as follows:
Absorbance at zero time=Absorbency as measured/weight of sample=A (say) which is taken as 100%.
Absorbency of any sample after exposure to UV light for specific time=Absorbency as measured/weight of sample=B (say).

The % absorbency remaining=B/A*100

The data is summarized in Table 1

TABLE 1

| | % absorbance remaining | | |
|---|---|---|---|
| Time (minutes) | Example 1 | Example 2 | Example A |
| 30 | 96 | 90 | 62 |
| 60 | 81 | 66 | 34 |
| 90 | 70 | 53 | 4 |
| 120 | 42 | 23 | 1 |

The data in Table-1 indicates that the composition where the UV-A or the UV-B sunscreen is encapsulated is significantly more stable as compared to the conventional compositions. Further the composition (Example 1) where the UV-A sunscreen is encapsulated is more stable as compared to the composition where UV-B is encapsulated.

### Example 3

A composition as per Example 1 was prepared except that both UV-A and UV-B were separately encapsulated. Parsol 1789 was encapsulated in 50% of the cetyl palmitate used in Example 1 while Parsol MCX was encapsulated in the balance 50% of the cetyl palmitate. The two slurries were cooled to 40° C. before they were mixed. The composition was then cooled to 25° C.

Composition of Example 3 was compared with Example 1 and comparative Example A by exposure to the sun at the same time and the data is summarized in Table 2.

TABLE 2

| | % absorbance remaining | | |
|---|---|---|---|
| Time (minutes) | Example 1 | Example 3 | Example A |
| 30 | 88 | 91 | 64 |
| 60 | 77 | 71 | 37 |
| 90 | 58 | 56 | 30 |
| 120 | 48 | 50 | 17 |

The data in Table 2 indicates that the composition where UV-A sunscreen is encapsulated in solid lipid encapsulates compares favorably with a composition where both UV-A and UV-B are separately encapsulated and then mixed. Both these compositions are significantly superior to the control sample prepared by the conventional method.

### Example 4

A composition as per Example 1 was prepared except that 0.8% antioxidant tocopherol acetate was additionally included in the solid lipid encapsulate. Also stearic acid was used as the lipid instead of cetyl palmitate.

### Example 5

A composition as per Example 2 was prepared except that 0.8% antioxidant tocopherol acetate was additionally included in the solid lipid encapsulate. Also stearic acid was used as the lipid instead of cetyl palmitate.

Samples of Example 4, Example 5 and comparative Example A were exposed for up to 120 minutes to the same conditions of sun exposure and the data is summarized in Table 3.

TABLE 3

| | % absorbance remaining | | |
|---|---|---|---|
| Time (minutes) | Example 4 | Example 5 | Example A |
| 30 | 95 | 77 | 55 |
| 60 | 76 | 53 | 36 |
| 90 | 75 | 36 | 19 |
| 120 | 58 | 36 | 17 |

The data in Table 3 indicates that improved stability is obtained by inclusion of an antioxidant in the solid lipid encapsulate.

### Example 6

A composition as per Example 4 was prepared except that butyl hydroxy tolune was used as an antioxidant in place of tocopherol acetate.

Samples of Example 4, Example 6 and comparative Example A were exposed for up to 60 minutes to the same conditions of sun exposure and the data is summarized in Table 4.

TABLE 4

| | % absorbance remaining | | |
|---|---|---|---|
| Time (minutes) | Example 4 | Example 6 | Example A |
| 15 | 83 | 92 | 71 |
| 30 | 79 | 78 | 60 |
| 45 | 74 | 76 | 44 |
| 60 | 82 | 80 | 39 |

The data in Table 4 indicates that other antioxidants could also be used in the solid lipid encapsulate to get enhanced stability of the UV-A sunscreen as compared to sunscreen composition of the prior art.

Efficacy of the Sunscreens

To ensure that the solid lipid encapsulate is transparent to the UV radiation thereby ensuring the efficacy of the sunscreens in absorbing at the desired wavelength ranges, experiments were conducted to measure the transmission of some samples.

An emulsion of 20% stearic acid in water was prepared (comparative Example B).

Samples of comparative Example A, B and Example 1 were taken on glass slides and film of about 2 mg/cm² was spread on them on an approximate area of about 6 cm².

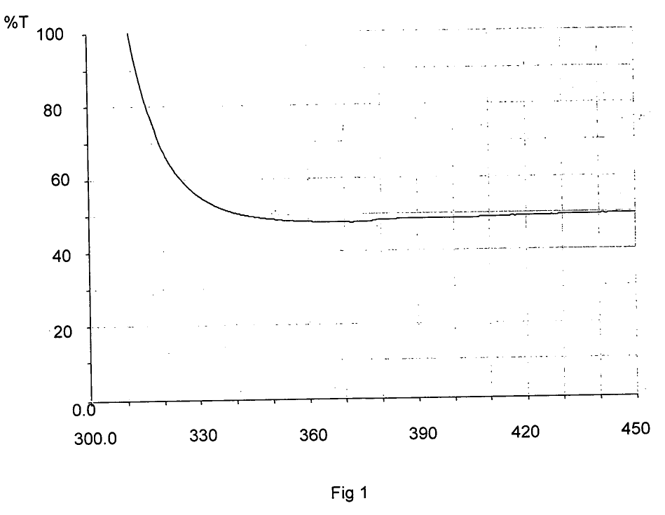

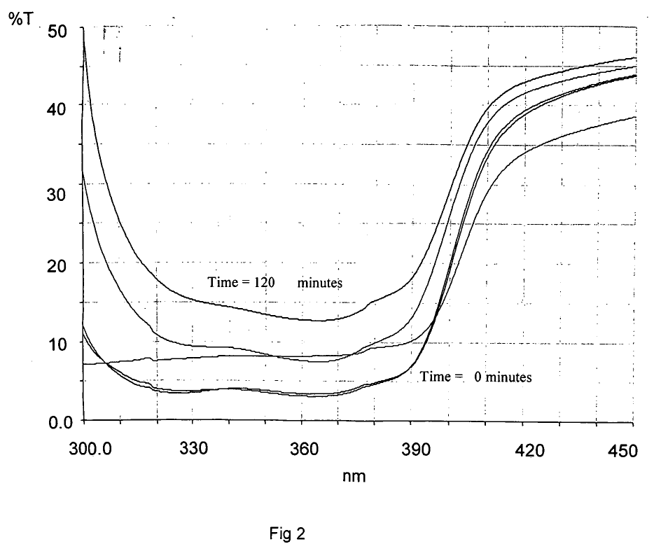

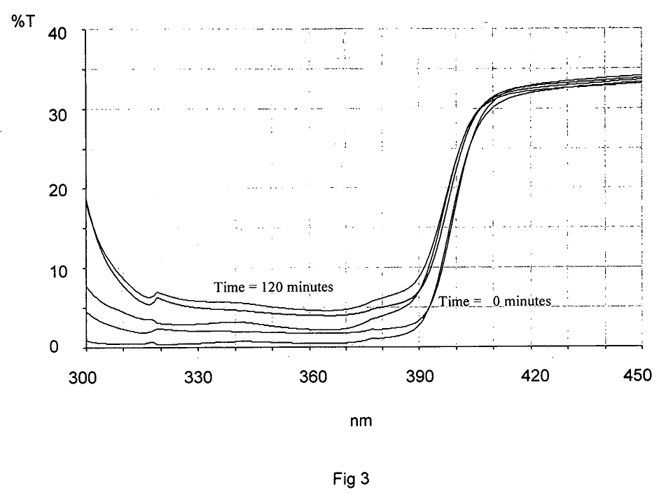

The transmittance of the samples were measured over a wavelength range of 300 to 450 nm using a Perkin Elmer (Lamda 900) UV/VIS/NIR spectrophotometer. The percentage transmittance as measured is shown in FIG. 1 (for comparative Example B), FIG. 2 (for comparative Example A) and FIG. 3 (for Example 1). The % transmittance was measured over a period of 120 minutes at various time intervals from 0 minutes to 120 minutes for Example 1 and comparative Example B. The data in FIGS. 1 to 3 indicate that a sample opaque to UV radiation (comparative Example B in FIG. 1) shows poor transmission over the UV wavelength range. FIG. 2 comprising UV-A and UV-B sunscreens which absorb the UV radiation show almost no transmission in the wavelength range of interest. FIG. 3 indicates that the transmission is comparable to that of a conventional composition (FIG. 2) thereby indicating the efficacy of the composition of the invention in selectively absorbing UV radiation. FIG. 3 also indicates that the composition of the invention is efficacious over a longer period of time as compared to a conventional composition.

Sunlamp Studies

The stability of the sunscreen compositions were studied under controlled UV radiation conditions. The samples of Example 5, Example 6 and comparative Example A were exposed to UV radiation using an Atlas MTT GmbH sunlamp at three different intensities viz. 25, 50 and 75 mW/cm² over a period of 90 minutes. Samples were taken every 30 minutes and the % absorbance remaining was measured. The data is summarized in Table 5.

TABLE 5

| | % absorbance remaining; | | | % absorbance remaining; | | | % absorbance remaining; | | |
|---|---|---|---|---|---|---|---|---|---|
| | 25 mW/cm² | | | 50 mW/cm² | | | 75 mW/cm² | | |
| Time | Example | Example | Example | Example | Example | Example | Example | Example | Example |
| (mins) | 5 | 6 | A | 5 | 6 | A | 5 | 6 | A |
| 15 | 85 | 78 | 68 | 86 | 86 | 67 | 81 | 77 | 50 |
| 30 | 80 | 80 | 49 | 72 | 86 | 52 | 74 | 70 | 34 |
| 45 | 70 | 69 | 41 | 74 | 77 | 36 | 56 | 58 | 25 |
| 60 | 71 | 69 | 36 | 77 | 72 | 38 | 51 | 46 | 12 |

The data in Table 5 indicates trends similar to that obtained under exposure of the samples to sunlight i.e the compositions comprising UV-A sunscreen, viz. Parsol 1789, are more stable when encapsulated in solid lipid along with an antioxidant, in compositions also comprising a UV-B sunscreen which is unencapsulated as compared to the compositions of the prior art.

The invention thus provides for a composition comprising an organic UV-A sunscreen and an organic UV-B sunscreen that have enhanced stability in use, compared to that reported in prior art. Further the enhanced stability is attained using conventional cosmetic ingredients and by using a simple and cost-effective process that ensures that the product is of low cost to the consumer.

### Example 7

This example is an anti-tanning test using human volunteers.

On day one, four test sites of 1 cm² were marked on both volar forearms of 15 volunteers using a template and 3 mg/cm² of the following test formulations were applied once a day on each marked part and left for 15 minutes. The samples tested were as follows:

Comparative Example A: A conventional sunscreen composition was prepared comprising both unencapsulated Parsol MCX (0.4% by weight) and unencapsulated Parsol 1789 (0.75% by weight).

Test Example: A sunscreen composition comprising 0.75% by weight of Parsol 1789 encapsulated in stearic acid and 0.4% by weight Parsol MCX dispersed in the composition.

The entire forearm was kept covered and only the test sites were exposed to 30 minutes of sunlight. This procedure was followed for three consecutive days. On day one, the tan caused by immediate pigment darkening (IPD) was measured on a visual scale between 0-8 where zero refers to no tan and 8 refers to deep tan by trained assessors 1 hour after exposure. Tan due to delayed tanning (DT) was also measured in the same way regularly for eight days. The tan protection index (TPI) was calculated using the following equation:
Tan protection index=Visual tan score of the control site−(visual tan score of the treated site/visual tan score of the control site).

TABLE 6

Tan protection index

| | Example A | Test Example |
|---|---|---|
| IPD | 0.00 | 0.21 |
| DT (after 8 days) | 0.12 | 0.40 |

A higher TPI indicates provision of superior protection and the data shows that considering both IPD and DT the composition comprising encapsulated Parsol 1789 (UV-A sunscreen) and unencapsulated Parcol MCX is significantly superior to Comparative Example A comprising the same sunscreens but with neither encapsulated.

1. A stable sunscreen composition comprising:
(i) an organic UV-A sunscreen;
(ii) an organic UV-B sunscreen; and
(iii) cosmetically acceptable vehicle
Wherein said UV-A sunscreen and UV-B sunscreen are kept isolated from each other by encapsulating one of said sunscreens in solid lipid encapsulate, the solid lipid encapsulate comprising a lipid.

2. A stable sunscreen composition according to claim 1 wherein both of said UV-A sunscreen and UV-B sunscreen are separately encapsulated in solid lipid encapsulates.

3. A stable sunscreen composition according to claim 1 wherein only UV-A sunscreen is encapsulated in solid lipid encapsulate.

4. A sunscreen according to claim 1 wherein the organic UV-A sunscreen is a dibenzoyl methane derivative, such as butylmethoxy dibenzoylmethane.

5. A sunscreen composition according to claim 1 wherein the UV-B sunscreen is octyl methoxycinnamate.

6. A sunscreen composition according to claim 1 wherein said solid lipid encapsulate comprises an emulsifier, such as a nonionic surfactant.

7. A sunscreen composition according to claim 1 wherein the solid lipid encapsulate comprises an antioxidant, such as tocopherol acetate or butyl hydroxyl toluene or ascorbyl palmitate.

8. A sunscreen composition according to claim 7 wherein said antioxidant is present in an amount in the range of 0.01 to 5% by weight of the composition.

9. A sunscreen composition according to claim 1 wherein said lipid is a fatty acid or fatty acid ester, such as glycerol monostearate or stearic acid or cetyl palmitate.

10. A sunscreen composition according to claim 1 wherein said UV-A sunscreen is present in an amount in the range of 0.1 to 5% by weight of the composition.

11. A sunscreen composition according to claim 1 wherein said UV-B sunscreen is present in an amount in the range of 0.1 to 5% by weight of the composition.

12. A sunscreen composition according claim 1 wherein said lipid in the solid lipid encapsulate is present in an amount in the range of 1 to 50% by weight of the composition.

13. A sunscreen composition according to claim 1 comprising a skin-lightening agent, such as niacinamide.

14. A sunscreen composition according to claim 13 wherein niacinamide is present in an amount in range of 0.1 to 10% by weight of the composition.

15. A process for preparation of the sunscreen composition according to claim 1 comprising the steps of:
(a) preparing a slurry by mixing the sunscreen to be encapsulated with the lipid at a temperature in the range of 60 to 90° C.;
(b) cooling said slurry to a temperature in the range of 30 to 50° C.;
(c) preparing a base by mixing the cosmetically acceptable vehicle and the sunscreen to be unencapsulated, and
(d) mixing the cooled slurry of step (b) with the base of step (c), at a temperature in the range of 30 to 50° C. to prepare the sunscreen composition.

16. A process according to claim 15 wherein an emulsion comprising antioxidant and emulsifier is mixed with said slurry at a temperature in the range of 60 to 90° C.
